(19) United States
(12) Patent Application Publication
(10) Pub. No.: US 2013/0261358 A1
(43) Pub. Date: Oct. 3, 2013

(54) METHODS AND APPARATUSES FOR ISOMERIZATION OF PARAFFINS

(71) Applicant: David James Shecterle, Arlington Heights, IL (US)

(72) Inventor: David James Shecterle, Arlington Heights, IL (US)

(73) Assignee: UOP LLC, Des Plaines, IL (US)

(21) Appl. No.: 13/434,657

(22) Filed: Mar. 29, 2012

(51) Int. Cl. C07C 5/27; B01J 19/00

(52) U.S. Cl. 585/264; 422/187

(57) ABSTRACT

Embodiments of methods and apparatuses for isomerization of paraffins are provided. In one example, a method comprises the steps of separating an isomerization effluent into a product stream that comprises branched paraffins and a stabilizer overhead vapor stream that comprises HCl, H₂, and C₆-hydrocarbons. C₆-hydrocarbons are removed from at least a portion of the stabilizer overhead vapor stream to form a HCl and H₂-rich stream. An isomerization catalyst is activated using at least a portion of the HCl and H₂-rich stream to form a chloride-promoted isomerization catalyst. A paraffin feed stream is contacted with the chloride-promoted isomerization catalyst in the presence of hydrogen for isomerization of the paraffins.

### TECHNICAL FIELD

The present invention relates generally to methods and apparatuses for isomerization of hydrocarbons, and more particularly relates to methods and apparatuses for isomerization of paraffins using a chloride-promoted isomerization catalyst.

### BACKGROUND

Isomerization processes are widely used by many refiners to rearrange the molecular structure of straight chain paraffinic hydrocarbons to more highly branched hydrocarbons that generally have higher octane ratings. Many isomerization processes employ a chlorinated catalyst, such as chlorinated alumina catalyst, chlorinated platinum aluminum catalyst, and the like, in a reaction zone (e.g., refers to an area including one or more reactors). The chlorinated catalyst requires a continuous addition of chloride to replace chloride removed from the surface of the catalyst and carried away in the reaction-zone effluent. Typically, a fresh feed of chloride promoter, such as perchloroethylene, is continuously introduced into a paraffin feed stream upstream from a reactor in the reaction zone. Inside the reactor, the chloride promoter decomposes to form hydrogen chloride that activates, e.g., promotes or regenerates, the catalyst by replenishing the chloride removed from the catalyst's surface.

The reaction-zone effluent generally contains a significant amount of hydrogen chloride from the continuous decomposition of chloride promoter and the removal of chloride from the surface of the catalyst. A product stream containing branched paraffins is separated from the reaction-zone effluent by removing hydrogen chloride and other light volatile hydrocarbons (e.g., C₅-hydrocarbons) as a stabilizer overhead vapor stream. Because hydrogen chloride poses environmental and handling concerns, the stabilizer overhead vapor stream is continuously scrubbed with a caustic, such as sodium hydroxide, to neutralize the hydrogen chloride before removing the off-gas stream from the process. The cost of chloride promoters and caustics are relatively expensive, and many refiners would like to reduce their consumption of these components to improve their process efficiencies and reduce overall operational costs.

Accordingly, it is desirable to provide methods and apparatuses for isomerization of paraffins with reduced chloride promoter consumption and/or reduced caustic consumption to improve process efficiencies and reduce overall operational costs. Furthermore, other desirable features and characteristics of the present invention will become apparent from the subsequent detailed description and the appended claims, taken in conjunction with the accompanying drawings and this background.

### BRIEF SUMMARY

Methods and apparatuses for isomerization of paraffins are provided herein. In accordance with an exemplary embodiment, a method for isomerization of paraffins comprises the steps of separating an isomerization effluent into a product stream that comprises branched and un-branched paraffins and a stabilizer overhead vapor stream that comprises HCl, H₂, and C₆-hydrocarbons. C₆-hydrocarbons are removed from at least a portion of the stabilizer overhead vapor stream to form a HCl and H₂-rich stream. An isomerization catalyst is activated using at least a portion of the HCl and H₂-rich stream to form a chloride-promoted isomerization catalyst. A paraffin feed stream is contacted with the chloride-promoted isomerization catalyst in the presence of hydrogen for isomerization of the paraffins.

In accordance with another exemplary embodiment, a method for isomerization of paraffins is provided. The method comprises the steps of introducing an isomerization effluent to a stabilizer at stabilization conditions effective to form a product stream that comprises branched and un-branched paraffins and a stabilizer overhead vapor stream that comprises HCl, H₂, and C₆-hydrocarbons. The stabilizer overhead vapor stream is separated in a first separator at first separation conditions into a liquid stream that comprises C₃⁺ hydrocarbon and a net gas stream that comprises HCl, H₂, and C₅-hydrocarbons. A C₅-hydrocarbons-rich stream and a HCl and H₂-rich stream that comprises HCl and H₂ are formed from at least a portion of the net gas stream in a second separator at second separation conditions. At least a portion of the HCl and H₂-rich stream is introduced to a reactor operating at isomerization conditions to activate an isomerization catalyst contained therein and form a chloride-promoted isomerization catalyst. A paraffin feed stream is contacted with the chloride-promoted isomerization catalyst in the reactor in the presence of hydrogen for isomerization of the paraffins.

In accordance with another exemplary embodiment, an apparatus for isomerization of paraffins is provided. The apparatus comprises a stabilizer configured to receive an isomerization effluent and to operate at stabilization conditions effective to separate the isomerization effluent into a product stream and a stabilizer overhead vapor stream. The product stream comprises branched and un-branched paraffins and the stabilizer overhead vapor stream comprises HCl, H₂, and C₅-hydrocarbons. A first separator is configured to receive the stabilizer overhead vapor stream and to operate at first separation conditions effective to separate the stabilizer overhead vapor stream into a liquid stream that comprises C₃⁺hydrocarbon and a net gas stream that comprises HCl, H₂, and C₅-hydrocarbons. A second separator is configured to receive at least a portion of the net gas stream and to operate at second separation conditions effective to separate the at least the portion of the net gas stream into a C₅-hydrocarbons-rich stream and a HCl and H₂-rich stream that comprises HCl and H₂. A reaction zone contains an isomerization catalyst. The reaction zone is configured to receive at least a portion of the HCl and H₂-rich stream and a paraffin feed stream and to operate at isomerization conditions to activate the isomerization catalyst to form a chloride-promoted isomerization catalyst. The chloride-promoted isomerization catalyst is for contact with the paraffin feed stream in the presence of hydrogen for isomerization of the paraffins.

### BRIEF DESCRIPTION OF THE DRAWINGS

The present invention will hereinafter be described in conjunction with the following drawing figures, wherein like numerals denote like elements, and wherein:

FIG. 1 schematically illustrates an apparatus and method for isomerization of paraffins in accordance with an exemplary embodiment.

### DETAILED DESCRIPTION

The following Detailed Description is merely exemplary in nature and is not intended to limit the invention or the application and uses of the invention. Furthermore, there is no intention to be bound by any theory presented in the preceding background or the following detailed description.

Various embodiments contemplated herein relate to methods and apparatuses for isomerization of paraffins. Unlike the prior art, the exemplary embodiments taught herein introduce an isomerization reaction-zone effluent from an isomerization reaction zone to a stabilizer. The isomerization reaction-zone effluent comprises HCl, H₂, branched paraffins, and C₇-hydrocarbons. As used herein, Cₓ means hydrocarbon molecules that have “X” number of carbon atoms, Cₓ+ means hydrocarbon molecules that have “X” and/or more than “X” number of carbon atoms, and Cₓ− means hydrocarbon molecules that have “X” and/or less than “X” number of carbon atoms. The stabilizer is operating at stabilization conditions effective to separate the isomerization reaction-zone effluent into a product stream that comprises the branched paraffins and a stabilizer overhead vapor stream that comprises HCl, H₂, and C₆-hydrocarbons.

Next, a portion of the C₆-hydrocarbons are removed from at least a portion of the stabilizer overhead vapor stream to form a HCl and H₂-rich stream. In an exemplary embodiment, C₆-hydrocarbons are removed from at least a portion of the stabilizer overhead vapor stream using a first separator and a second separator that is in fluid communication with the first separator. In particular, the stabilizer overhead vapor stream is introduced to the first separator at separation conditions effective to form a liquid stream that comprises C₃⁺ hydrocarbon and a net gas stream that comprises HCl and C₅-hydrocarbons. The liquid stream is directed back to the stabilizer for reflux. In an exemplary embodiment, the net gas stream is divided into a first portion and a second portion. The first portion of the net gas stream is introduced to the second separator at separation conditions effective to form a C₅-hydrocarbons-rich stream and a HCl and H₂-rich stream. The C₅-hydrocarbons-rich stream, which is substantially depleted of HCl, is heated and combined with the second portion of the net gas stream to form a combined stream that is directed to a scrubber for treatment with a caustic. Because the C₅-hydrocarbons-rich stream is substantially free of HCl, the amount of HCl in the combined stream is reduced and thus, less caustic is required to neutralize the HCl. Therefore, caustic consumption can be reduced for the isomerization process.

In an exemplary embodiment, at least a portion of the HCl and H₂-rich stream is introduced to a reactor in the isomerization reaction zone. The reactor contains an isomerization catalyst and is operating at isomerization conditions. The isomerization catalyst is contacted with the HCl and H₂-rich stream to activate the isomerization catalyst by replenishing chloride removed from the surface of the isomerization catalyst, forming a chloride-promoted isomerization catalyst. Because the HCl and H₂-rich stream is used to activate the isomerization catalyst, less chloride promoter is required for activating the isomerization catalyst. Therefore, chloride promoter consumption can be reduced for the isomerization process. Since H₂ is also contained in the HCl and H₂-rich stream, less makeup hydrogen is required and hydrogen consumption is reduced. A feed stream containing paraffins is introduced to the reactor and contacts the chloride-promoted isomerization catalyst in the presence of hydrogen to isomerize the paraffins and form branched paraffins.

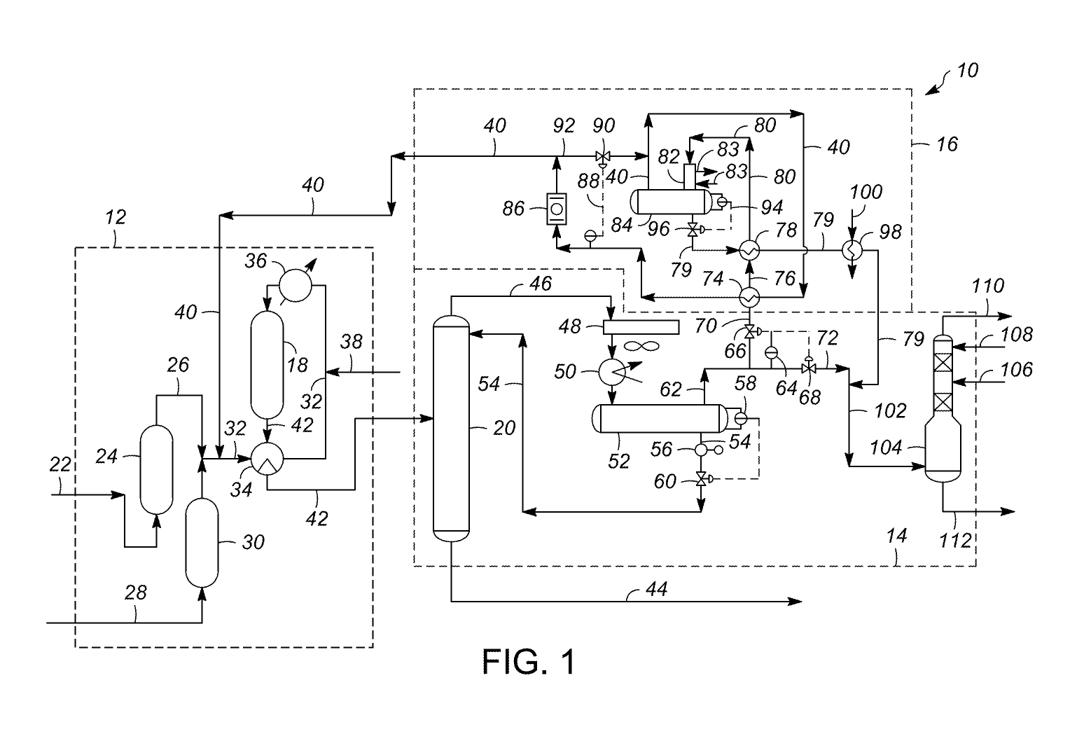

Referring to FIG. 1, a schematic depiction of an apparatus 10 for isomerization of paraffins is provided. The apparatus 10 is utilized for a paraffin isomerization process that converts normal paraffins to branched paraffins. The apparatus 10 comprises a reaction zone 12, a stabilizing-scrubbing zone 14, and a separation-recycle zone 16.

The reaction zone 12 and the stabilizing-scrubbing zone 14 include a reactor 18 and stabilizer 20 (e.g., distillation column), respectively, that are in fluid communication. A paraffin feed stream 22 is passed through a dryer 24 for removing water and to form a dried paraffin feed stream 26. In one embodiment, the paraffin feed stream 22 is rich in C₄ hydrocarbons, such as n-butane and may also contain relatively small amounts of iso-butane, pentane, and heavier materials (e.g., C₆⁺ hydrocarbons). In another embodiment, the paraffin feed stream 22 is rich in C₅ and/or C₆ hydrocarbons, such as normal pentane and normal hexane.

In an exemplary embodiment, a hydrogen-containing gas feed 28 is passed through a dryer 30 for removing water and is combined with the dried paraffin feed 26 to form a combined stream 32. The combined stream 32 is passed through a heat exchanger 34 and a heater 36. As illustrated and will be discussed in further detail below, a chloride promoter stream 38 (e.g., containing perchloroethylene or the like) is introduced to the combined stream 32 between the heat exchanger 34 and the heater 36, and a HCl and H₂-rich stream 40 (e.g., containing about 0.1 weight percent (wt. %) or greater of HCl) is introduced to the combined stream 32 upstream from the heat exchanger 34. In an exemplary embodiment, the heat exchanger 34 and the heater 36 together heat the combined stream 32 to a temperature of from about 90 to about 210° C. for introduction to the reactor 18.

In an exemplary embodiment, the reactor 18 is a fixed-bed catalytic reactor operating at a temperature of from about 90 to about 210° C. and contains an isomerization catalyst that is activated by HCl from the HCl and H₂-rich stream 40 and further, by the decomposition of chloride promoter from the chloride promoter stream 38 to form a high-activity chloride-promoted isomerization catalyst. Non-limiting examples of the isomerization catalyst include alumina catalyst, platinum aluminum catalyst, and the like that can be chlorinated. The chloride-promoted isomerization catalyst in the presence of hydrogen is effective to isomerize the normal paraffins to branched paraffins (e.g., iso-butane, branched pentane, branched hexane, or combinations thereof) to produce an isomerization reaction-zone effluent 42. The isomerization reaction-zone effluent 42 contains the branched paraffins, other C₇-hydrocarbons including un-branched paraffins, H₂, HCl, and possibly other chloride-containing compounds.

The isomerization reaction-zone effluent 42 is passed through the heat exchanger 34 to cool the effluent 42 to a temperature of from about 65 to about 165° C. The isomerization reaction-zone effluent 42 is then introduced to the stabilizer 20. The stabilizer 20 separates the isomerization reaction-zone effluent 42 into a product stream 44 and a stabilizer overhead vapor stream 46. The stabilizer overhead vapor stream 46 contains HCl, H₂, and C₆-hydrocarbons. The product stream 44 contains branched and un-branched paraffins and is removed from the stabilizing-scrubbing zone 14.

In an exemplary embodiment, the stabilizer overhead vapor stream 46 is passed through an air cooler 48 and a partial condenser 50 that together cool the stabilizer overhead vapor stream 46 to a temperature of from about 30 to about 60° C. The stabilizer overhead vapor stream 46 is then introduced to a separator 52 for separation. A liquid stream 54 containing C₃⁺ hydrocarbon is removed from the separator 52 and is passed through a pump 56. A level controller 58 including a control valve 60 controls the flow of the liquid stream 54 back to the stabilizer 20 for reflux.

Volatiles are removed from the separator 52 and form a net gas stream 62. The net gas stream 62 contains HCl, H₂, and C₅-hydrocarbons. In an exemplary embodiment, the net gas stream 62 is removed from the separator 52 at a pressure of from about 700 to about 2,100 kPa. As illustrated, a pressure controller 64 along with control valves 66 and 68 are used to divide the net gas stream 62 into portions 70 and 72, respectively.

In an exemplary embodiment, the portion 70 of the net gas stream 62 is introduced to the separation-recycle zone 16. As illustrated, the portion 70 is passed through a heat exchanger 74 for indirect heat exchange with the HCl and H₂-rich stream 40 to form a first partially-cooled net gas stream 76. In an exemplary embodiment, the heat exchanger 74 cools the portion 70 to form the first partially-cooled net gas stream 76 that has a temperature of from about −7 to about 38° C.

The first partially-cooled net gas stream 76 is passed through a heat exchanger 78 for indirect heat exchange with a C₅-hydrocarbons-rich stream 79 (discussed in further detail below) to form a second partially-cooled net gas stream 80. In an exemplary embodiment, the heat exchanger 78 cools the first partially-cooled net gas stream 76 to form the second partially-cooled net gas stream 80 that has a temperature of from about −18 to about 32° C.

The second partially-cooled net gas stream 80 is then passed through a heat exchanger 82 for indirect heat exchange with a refrigerant 83, e.g., propane or the like, and is introduced to a separator 84. As illustrated, the heat exchanger 82 may be mounted directly on the separator 84, or alternatively, may be positioned upstream from the separator 84. In an exemplary embodiment, the heat exchanger 82 further cools the second partially-cooled net gas stream 80 to a temperature of from about −40 to about 5° C. before introduction to the separator 84.

The separator 84 separates the second partially-cooled net gas stream 80 into the HCl and H₂-rich stream 40 and the C₅-hydrocarbons-rich stream 79. In an exemplary embodiment, the HCl and H₂-rich stream 40 comprises HCl present in an amount of about 0.1 wt. % or greater, such as about from 0.2 to 0.7 wt. %, and H₂. In an exemplary embodiment, the C₅-hydrocarbons-rich stream 79 contains primarily C₅-hydrocarbons and is substantially depleted of HCl and H₂.

As illustrated, the HCl and H₂-rich stream 40 is passed through the heat exchanger 74 for indirect heat exchange with the portion 70 of the net gas stream 62 as discussed above. In an exemplary embodiment, the heat exchanger 74 heats the HCl and H₂-rich stream 40 to a temperature of from about −7 to about 25° C. The HCl and H₂-rich stream 40 is then passed through a compressor 86. In an exemplary embodiment, the compressor 86 pressurizes the HCl and H₂-rich stream 40 to a pressure of from about 1,700 to about 3,500 kPa.

As illustrated, a flow controller 88 including a control valve 90 is used to selectively divert a spillback stream 92 from the HCl and H₂-rich stream 40 as needed (.e.g., normally the spillback stream 92 is “no flow”). The spillback stream 92, when flowing, is pressurized and returned to the HCl and H₂-rich stream 40 upstream from the heat exchanger 74. The remaining portion of the HCl and H₂-rich stream 40 is passed along from the compressor 86 and is combined with the combined streams 32 for introduction to the reactor 18 together with the chloride promoter stream 38. As discussed above, once introduced to the reactor 18, HCl from the from the HCl and H₂-rich stream 40 and further from the decomposition of chloride promoter from the chloride promoter stream 38 contacts and activates the isomerization catalyst by replenishing chloride removed from the surface of the isomerization catalyst. Because the HCl and H₂-rich stream 40 is used to activate the isomerization catalyst, less chloride promoter is required from the chloride promoter stream 38 for activating the isomerization catalyst.

As illustrated, a level controller 94 including a control valve 96 is used to control the flow of the C₅-hydrocarbons-rich stream 79 leaving the separator 84. The C₅-hydrocarbons-rich stream 79 is passed through the heat exchanger 78 for indirect heat exchange with the first partially-cooled net gas stream 76 as discussed above. In an exemplary embodiment, the C₅-hydrocarbons-rich stream 79 is heated to a temperature of from about −18 to about 15° C.

The C₅-hydrocarbons-rich stream 79 is then passed through a heat exchanger 98 for indirect heat exchange with a heat transfer fluid 100, such as steam. In an exemplary embodiment, the heat exchanger 98 heats the C₅-hydrocarbons-rich stream 79 to a temperature of from about 30 to about 70° C. The C₅-hydrocarbons-rich stream 79 is passed from the heat exchanger 98 and combined with the portion 72 of the net gas stream 62 to form a combined stream 102. As illustrated, the combined stream 102 is introduced to a scrubber 104. The scrubber 104 scrubs the combined stream 102 by neutralizing any HCl contained therein with a caustic 106 followed by counter flow contact with water 108 to form a neutralized stream 110 and a caustic waste stream 112.

Accordingly, methods and apparatuses for isomerization of paraffins have been described. The exemplary embodiments taught herein introduce an isomerization reaction-zone effluent from an isomerization reaction zone to a stabilizer. The isomerization reaction-zone effluent comprises HCl, H₂, branched paraffins, and C₇-hydrocarbons. The stabilizer separates the isomerization reaction-zone effluent into a product stream that comprises the branched paraffins and a stabilizer overhead vapor stream that comprises HCl and C₆-hydrocarbons. The stabilizer overhead vapor stream is introduced to a first separator to remove C₃⁺ hydrocarbons and form a net gas stream that comprises HCl, H₂, and C₅-hydrocarbons. The net gas stream is divided into a first portion and a second portion. The first portion is introduced to the second separator to form a C₅-hydrocarbons-rich stream and a HCl and H₂-rich stream. The C₅-hydrocarbons-rich stream, which is substantially depleted of HCl, is heated and combined with the second portion to form a combined stream that is directed to a scrubber for treatment with a caustic. Because the C₅-hydrocarbons-rich stream is substantially free of HCl, the amount of HCl in the combined stream is reduced and thus, less caustic is required to neutralize the HCl. The HCl and H₂-rich stream is introduced to a reactor in the isomerization reaction zone. The reactor contains an isomerization catalyst that is contacted with the HCl and H₂-rich stream to form a chloride-promoted isomerization catalyst. Because the HCl and H₂-rich stream is used to activate the isomerization catalyst, less chloride promoter is required for activating the isomerization catalyst.

While at least one exemplary embodiment has been presented in the foregoing detailed description of the disclosure, it should be appreciated that a vast number of variations exist. It should also be appreciated that the exemplary embodiment or exemplary embodiments are only examples, and are not intended to limit the scope, applicability, or configuration of the disclosure in any way. Rather, the foregoing detailed description will provide those skilled in the art with a convenient road map for implementing an exemplary embodiment of the disclosure. It being understood that various changes may be made in the function and arrangement of elements described in an exemplary embodiment without departing from the scope of the disclosure as set forth in the appended claims.

1. A method for isomerization of paraffins, the method comprising the steps of:
separating an isomerization effluent into a product stream that comprises branched and un-branched paraffins and a stabilizer overhead vapor stream that comprises HCl, H₂, and C₆-hydrocarbons;
removing C₆-hydrocarbons from at least a portion of the stabilizer overhead vapor stream to form a HCl and H₂-rich stream;
activating an isomerization catalyst using at least a portion of the HCl and H₂-rich stream to form a chloride-promoted isomerization catalyst; and
contacting a paraffin feed stream with the chloride-promoted isomerization catalyst in the presence of hydrogen for isomerization of the paraffins.

2. The method of claim 1, wherein the step of separating comprises separating the isomerization effluent at a stabilizer temperature of from about 65 to about 165° C.

3. The method of claim 1, wherein the step of removing comprises separating the stabilizer overhead vapor stream at first separation conditions effective to form a liquid stream that comprises C₃⁺ hydrocarbon and a net gas stream that comprises HCl, H₂, and C₅-hydrocarbons.

4. The method of claim 3, wherein the step of removing comprises separating the stabilizer overhead vapor stream at the first separation conditions that include a first separation temperature of from about 30 to about 60° C.

5. The method of claim 3, wherein the step of removing comprises separating the stabilizer overhead vapor stream at the first separation conditions that include a first separation pressure of from about 700 to about 2,100 kPa gauge.

6. The method of claim 3, wherein the step of removing comprises:
dividing the net gas stream into a first portion and a second portion; and
removing C₅-hydrocarbons from the first portion of the net gas stream to form the HCl and H₂-rich stream.

7. The method of claim 6, further comprising:
neutralizing the second portion of the net gas stream with a caustic.

8. The method of claim 3, wherein the step of removing comprises separating at least a portion of the net gas stream at second separation conditions effective to form a C₅-hydrocarbons-rich stream and the HCl and H₂-rich stream.

9. The method of claim 8, wherein the step of removing comprises separating the at least the portion of the net gas stream at the second separation conditions that include a second separation temperature of from about −40 to about 5° C.

10. The method of claim 8, wherein the step of removing comprises separating the at least the portion of the net gas stream at the second separation conditions that include a second separation pressure of from about 1,200 to about 2,100 kPa gauge.

11. A method for isomerization of paraffins, the method comprising the steps of:
introducing an isomerization effluent to a stabilizer at stabilization conditions effective to form a product stream that comprises branched and unbranched paraffins and a stabilizer overhead vapor stream that comprises HCl, H₂, and C₆-hydrocarbons;
separating the stabilizer overhead vapor stream in a first separator at first separation conditions into a liquid stream that comprises C₃⁺ hydrocarbon and a net gas stream that comprises HCl, H₂, and C₅-hydrocarbons;
forming a C₅-hydrocarbons-rich stream and a HCl and H₂-rich stream that comprises HCl and H₂ from at least a portion of the net gas stream in a second separator at second separation conditions;
introducing at least a portion of the HCl and H₂-rich stream to a reactor operating at isomerization conditions to activate an isomerization catalyst contained therein and form a chloride-promoted isomerization catalyst; and
contacting a paraffin feed stream with the chloride-promoted isomerization catalyst in the reactor in the presence of hydrogen for isomerization of the paraffins.

12. The method of claim 11, further comprising:
introducing the at least the portion of the net gas stream to a first heat exchanger for indirect heat exchange with the HCl and H₂-rich stream to cool the at least the portion of the net gas stream to a first temperature prior to being introduced to the second separator.

13. The method of claim 12, wherein the step of introducing the at least a portion of the net gas stream to the first heat exchanger comprises cooling the at least the portion of the net gas stream to the first temperature of from about −7 to about 38° C.

14. The method of claim 11, further comprising:
introducing the at least the portion of the net gas stream to a second heat exchanger for indirect heat exchange with the C₅-hydrocarbons-rich stream to cool the at least the portion of the net gas stream to a second temperature prior to being introduced to the second separator.

15. The method of claim 14, wherein the step of introducing the at least the portion of the net gas stream to the second heat exchanger comprises cooling the at least the portion of the net gas stream to the second temperature of from about −18 to about 32° C.

16. The method of claim 14, further comprising:
dividing the net gas stream into a first portion and a second portion, and wherein the step of introducing the at least the portion of the net gas stream to the second heat exchanger comprises introducing the first portion of the net gas stream to the second heat exchanger;
combining the C₅-hydrocarbons-rich stream with the second portion of the net gas stream to form a combined stream after introducing the first portion of the net gas stream to the second heat exchanger; and
introducing the combined stream to a scrubber to neutralize HCl with a caustic.

17. The method of claim 11, further comprising:
introducing the at least the portion of the net gas stream to a third heat exchanger for indirect heat exchange with a refrigerant to cool the at least the portion of the net gas stream to a third temperature prior to being introduced to the second separator.

18. The method of claim 17, wherein the step of introducing the at least the portion of the net gas stream to the third heat exchanger comprises cooling the at least the portion of the net gas stream to the third temperature of from about −40 to about 5° C.

19. An apparatus for isomerization of paraffins, the apparatus comprising:
a stabilizer configured to receive an isomerization effluent and to operate at stabilization conditions effective to separate the isomerization effluent into a product stream that comprises branched and un-branched paraffins and a stabilizer overhead vapor stream that comprises HCl, H₂, and C₆-hydrocarbons;
a first separator configured to receive the stabilizer overhead vapor stream and to operate at first separation conditions effective to separate the stabilizer overhead vapor stream into a liquid stream that comprises C₃⁺ hydrocarbon and a net gas stream that comprises HCl, H₂, and C₅-hydrocarbons;
a second separator configured to receive at least a portion of the net gas stream and to operate at second separation conditions effective to separate the at least the portion of the net gas stream into a C₅-hydrocarbons-rich stream and a HCl and H₂-rich stream that comprises HCl and H₂; and
a reaction zone containing an isomerization catalyst and configured to receive at least a portion of the HCl and H₂-rich stream and a paraffin feed stream and to operate at isomerization conditions to activate the isomerization catalyst to form a chloride-promoted isomerization catalyst for contact with the paraffin feed stream in the presence of hydrogen for isomerization of the paraffins.

20. The apparatus of claim 19, further comprising:
at least one heat exchanger in fluid communication with the first and second separators and including a first heat exchanger, a second heat exchanger, and/or a third heat exchanger, wherein the first heat exchanger is configured to receive the at least the portion of the net gas stream for indirect heat exchange with the HCl and H₂-rich stream to cool the at least the portion of the net gas stream to a first temperature prior to being introduced to the second separator, wherein the second heat exchanger is configured to receive the at least the portion of the net gas stream for indirect heat exchange with the C₅-hydrocarbons-rich stream to cool the at least the portion of the net gas stream to a second temperature prior to being introduced to the second separator, and wherein the third heat exchanger is configured to receive the at least the portion of the net gas stream for indirect heat exchange with a refrigerant to cool the at least the portion of the net gas stream to a third temperature prior to being introduced to the second separator.
